(19) United States
(12) Patent Application Publication
(10) Pub. No.: US 2023/0218479 A1
(43) Pub. Date: Jul. 13, 2023

(54) MASSAGER HEAD

(71) Applicant: HUIZHOU YINGYI MOTOR CO., LTD., Guangdong (CN)

(72) Inventor: Chengyi HU, Guangdong (CN)

(21) Appl. No.: 17/621,615

(22) PCT Filed: Mar. 13, 2020
(86) PCT No.: PCT/CN2020/079315

§ 371 Date: Dec. 21, 2021

(30) Foreign Application Priority Data
Jun. 27, 2019 (CN) 201920985067.3

(51) Int. Cl. A61H 37/00

(52) U.S. Cl. CPC A61H 37/00; A61H 2201/0207; A61H 2201/0214; A61H 2201/0257

(57) ABSTRACT

A massager head comprising a massager main body, a contact headarranged on the massager main body for contacting a user’s skin, muscles, fascia and other surfaces, and a temperature control part arranged in the massager main body and connected with the contact head. The massager head is designed in a flat plate to better take care of the skin, muscles, fascia and the like, and added with the temperature control part for cooling or heating so that the massager head can be used at a relatively hot or cold temperature, which brings a better use effect to the user.

### TECHNICAL FIELD

The invention relates to the field of massage instrument heads, in particular to a massage instrument head.

### BACKGROUND TECHNIQUE

With the improvement of people’s living standards, people are more pursuing the quality of life. Especially for skin, muscle, fascia, etc. care and body massage. There are many massage devices on the market for skin, muscle, fascia, etc. care and body massage. However, most of the massage device heads are simple spherical structures and cannot better care for skin, muscles, fascia, etc. and massage. At the same time, the temperature of the existing massager head cannot be set, which will affect the effect of use when it is hot or cold.

### SUMMARY OF THE INVENTION

### TECHNICAL PROBLEM

The technical problem solved by the present invention is to provide a massage instrument head, which solves the problems that the existing massage instrument head cannot better care for the skin, muscles, fascia, etc. and cannot set the temperature, which affects the use effect when used in hot or cold temperatures.

In order to solve the above technical problems, the technical solution of the present invention is: a massage instrument head, including a massage instrument main body, on the massage instrument main body is provided with a contact head for contacting the user’s skin, muscles, fascia, etc. A temperature adjustment control is arranged in the main body of the massager, and the temperature adjustment control is connected with the contact head.

Preferably, the temperature adjustment control includes a cooling and heating fin and a heat dissipation plate, and the cooling and heating fin and the heat dissipation plate are closely attached.

Preferably, the cooling and heating fins are semiconductor cooling and heating fins.

Preferably, the heat dissipation plate is an aluminum alloy heat dissipation plate.

Preferably, a massage head protective cover is provided at the tail of the main body of the massager.

Preferably, the massage head protective cover is a massage head silicone cover

Preferably, the contact head is in the shape of a flat plate, and the contact head is a metal contact head, a plastic contact head or a silicone contact head.

Preferably, the metal contact head is an aluminum alloy contact head.

The beneficial effects achieved by the present invention: Compared with the existing massage instrument head, the massage instrument head of the present invention adopts a flat plate-like design, which can better care for the skin, muscles, fascia, etc., and has added temperature control controls, which can be cooled or cooled. Heating, so that it can be used in hot or cold temperature, to bring better use effect to users.

The solution to the problem

The beneficial effects of the invention

Brief description of the drawings

### DESCRIPTION OF THE DRAWINGS

FIG. 1 is a three-dimensional view of the massager head of the present invention.

FIG. 2 is an exploded view of the massager head of the present invention.

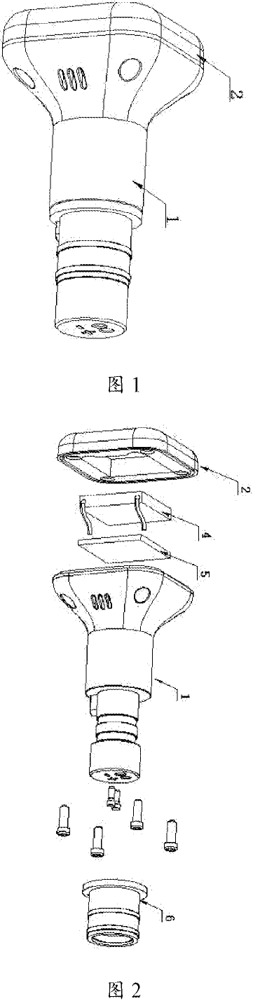

The numbers or letters in the figure represent the names or process names of the corresponding parts: 1. Massager main body; 2. Contact head; 3. Temperature adjustment control; 4. Cooling and heating fin; 5. Heat sink; 6. Massage head protection set.

The attached drawings are only for illustrative purposes and cannot be understood as a limitation of this patent; in order to better illustrate this embodiment, some parts of the attached drawings may be omitted, enlarged or reduced, and do not represent the size of the actual product; For the personnel, it is understandable that some well-known structures and their descriptions in the drawings may be omitted; the same or similar reference numerals correspond to the same or similar components; the terms describing the positional relationship in the drawings are only for exemplary description and cannot be understood. It is a limitation of this patent.

### INVENTION EMBODIMENT

### EMBODIMENTS OF THE PRESENT INVENTION

In order to facilitate the understanding of those skilled in the art, the present invention will be further described in detail below with reference to the accompanying drawings and embodiments. details as follows:

The massager head includes a massager main body 1. The massager main body 1 is provided with a contact head 2 for contacting the user’s skin, muscles, fascia, etc., and a temperature adjustment control 3 is provided in the massager main body 1. The piece 3 and the contact head 2 are connected.

Specifically, the temperature adjustment control 3 includes a cooling and heating fin 4 and a radiating plate 5, and the cooling and heating fin 4 and the radiating plate 5 are closely attached. In this embodiment, the cooling and heating fin is a semiconductor cooling and heating fin, and the radiating plate is an aluminum alloy radiating plate. By connecting the power supply, the cooling and heating fins can be heated or cooled, because the contact head is connected with the cooling and heating fin, and heat can be transferred to the contact head, thereby realizing the adjustment of the temperature.

Specifically, a massage head protective cover 6 is provided at the rear of the massage device main body 1. In this embodiment, the massage head protective cover 6 is a massage head silicone cover.

Specifically, the contact head is in the shape of a flat plate, and the contact head 2 is a metal contact head, a plastic contact head or a silicone contact head. In this embodiment, the metal contact head is an aluminum alloy contact head.

Obviously, the above-mentioned embodiments of the present invention are merely examples to clearly illustrate the present invention, and are not intended to limit the implementation of the present invention. For those of ordinary skill in the art, other changes or modifications in different forms can be made on the basis of the above description. It is not necessary and impossible to list all the implementation methods here. Any modification, equivalent replacement and improvement made within the spirit and principle of the present invention shall be included in the protection scope of the claims of the present invention.

1. A massage instrument head, characterized in that it comprises a massage instrument main body (1), and a contact head (2) for contacting the surface of the user’s skin, muscle or fascia is provided on the massage instrument main body (1). The massage instrument main body (1) is provided with a temperature adjustment control (3), and the temperature adjustment control (3) is connected to the contact head (2).

2. The massage instrument head according to claim 1, characterized in that: the temperature adjustment control (3) comprises a cooling and heating fin (4) and a heat dissipation plate (5), the cooling and heating fin (4) and the heat dissipation plate (5) fits tightly.

3. The massage instrument head according to claim 2, wherein the cooling and heating fin (4) is a semiconductor cooling and heating fin.

4. The massage instrument head according to claim 2, wherein the heat dissipation plate (5) is an aluminum alloy heat dissipation plate.

5. The massage instrument head according to claim 1, characterized in that a massage head protective cover (6) is provided at the tail of the massage instrument main body (1).

6. The massage instrument head according to claim 5, characterized in that: the massage head protective cover (6) is a massage head silicone cover.

7. The massage instrument head according to any one of claims 1-6, characterized in that: the contact head (2) is a flat plate shape, and the contact head (2) is a metal contact head, a plastic contact head or a silica gel Contact head.

8. The massage instrument head according to claim 7, wherein the metal contact head is an aluminum alloy contact head.
